(19) United States
(12) Patent Application Publication
(10) Pub. No.: US 2015/0139901 A1
(43) Pub. Date: May 21, 2015

(54) Radioactive Rhodium Complexes, Preparation Methods and Uses Thereof

(71) Applicants: Institut National de la Sante et de la Recherche Medicale (INSERM), Paris (FR); CHU Nantes, Nantes Cedex 1 (FR); Universite de Nantes, Nantes (FR); Centre National de la Recherche Scientifique (C.N.R.S.), Paris (FR); Universite d'Angers, Angers (FR)

(72) Inventors: Alain Faivre-Chauvet, Nantes Cedex 1 (FR); Holisoa Rajaerison, Nantes Cedex 1 (FR); Jean-Francois Gestin, Nantes Cedex 1 (FR)

(73) Assignees: Institut National de la Sante et de la Recherche (INSERM), Paris (FR); Chu Nantes, Nantes Cedex (FR); Universite de Nantes, Nantes (FR); Centre National de la Recherche Scientifique (C.N.R.S.), Paris (FR); Universite d' Angers, Angers (FR)

(21) Appl. No.: 14/399,305

(22) PCT Filed: May 14, 2013
(86) PCT No.: PCT/EP2013/059958

§ 371 Date: Nov. 6, 2014

(30) Foreign Application Priority Data
May 15, 2012 (EP) 12305543.6

(51) Int. Cl. C07F 15/00; A61K 47/48

(52) U.S. Cl. CPC C07F 15/0073; A61K 47/48092
USPC 424/1.65; 548/103; 514/184; 534/10

(57) ABSTRACT

The present invention concerns radioactive rhodium complexes, their preparation methods, and their use for the radiolabelling of biomolecules, especially monoclonal antibodies.

The present invention concerns radioactive rhodium complexes, their preparation methods, and their use for the radiolabelling of biomolecules, especially monoclonal antibodies.

Astatine-211 is a promising radionuclide for targeted alpha-therapy, which allows high radiation dose in small tumor volume while not affecting the surrounding healthy tissues. In association with a suited tumor-targeting biomolecule, its radiophysical properties make it one of the best candidates for the treatment of cancer. Particularly, its physical half-life (7.21 h) is adapted to the pharmacokinetics of biomolecules to be labelled for radiotherapy (Zalutsky M R, Vaidyanathan G (2000) Curr Pharm Des. 6; 1433-1455). It is produced by bombardment of alpha particles on bismuth-209 via the Bi-209(α,2n)At-211 nuclear reaction.

Astatine is the heaviest halogen. Because there is no stable isotope of this element and because the longest-lived isotope has only an 8.1 h half-life (At-210), its chemistry is not fully understood. In addition, only few cyclotrons can produce astatine-211. Thus, iodine is generally used to study and predict astatine reactivity. Iodine-125 is easily available and particularly useful in this context. However if some similarities are observed in the chemistry of astatine and iodine, in many aspects, there are noticeable differences (e.g. metallic properties for astatine) and preliminary results obtained with iodine may not be reproduced with astatine under similar conditions.

Several oxidation states of astatine have been established (−1, 0, +1, +3, +5, +7). For biomolecule labelling, At-211 is generally linked to the molecular vector in the +1 oxidation state (Aromatic carbon-astatine (Zalutsky M R, Pradeep K. Garg, Henry S. Friedman, and Darell D. Bigner (1989) Proc. Natl. Acad. Sci. U.S.A, 86, 7149-7153) to form carbon-astatine or boron-astatine bonds (Wilbur D S, Chyan M K, Hamlin D K, Perry M A. (2009) Bioconjugate Chem. 20; 591-602)) and less frequently in the −1 oxidation state (e.g.metal-astatine bond) (Pruszyfiski M, Bilewicz A, Zalutsky M R (2007) Bioconjugate Chem. 19; 958-965).

Astatine-211 is considered for targeted radionuclide therapy of various cancers after conjugation to a molecular vector. However deastatination of the molecular vector labelled with this atom has been observed in vivo, leading to non-specific irradiation of healthy organs. Improved labelling methods remain necessary to increase the stability of the astatine bond to its molecular vector.

The labelling methods developed for astatine can also find applications with radioactive isotopes of iodine. The most often considered isotope for therapy is iodine-131. It is a beta particle emitter with an 8 day half-life. It decays to the stable xenon-131. In association with suitable vectors, iodine-131 has already found clinical applications for cancer therapies (Macklis M R (2006) Int. J. Radiation Oncology Biol. Phys. 66; S30-S34). Iodine-125 which is easily available is generally used for preliminary radiolabelling tests before the use of the more expensive isotopes cited above. Its use is considered for therapy regarding its extremely short Auger electron emission especially when linked to a cell internalizing vector (Meredith M R et al (1995) J. Nucl. Med. 36; 2229-2233).

Iodine-123 and iodine-124 represent the most useful iodine isotopes for cancer detection. With a 13.2 h half-life and gamma decay, iodine-123 is suitable for various diagnostic applications by gamma camera detection (Bourguignon M H, Pauwels E K J, Loc'h C, Maziére B (1997) Eur. J. Nucl. Med. 24; 331-344). Iodine-124 decays by positron emission with a 4.2 day half-life. It can be used as a tracer in positron emission tomography (PET) (Pentlow K S et al (1996) J. Nucl. Med. 37; 1557-1562).

Therefore, there is a need to provide new stable complexes allowing the radiolabelling of biomolecules with radioactive isotopes of halogens.

The object of the present invention is to provide new radioactive complexes for the radiolabelling of biomolecules, especially monoclonal antibodies.

The object of the present invention is to provide radioactive halogen complexes that allow labelled biomolecules to remain labelled and bind specific organs to be detected or irradiated by using the halogen linked to a rhodium atom in a stabilised +1 oxidation state.

Another object of the present invention is to provide a method of preparation of these complexes.

Another object of the present invention is to provide a method of preparation of these complexes in good yields.

The present invention thus relates to a compound having the formula (I)

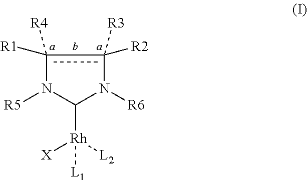

in which:

b is either a single or a double bond;
when b is a double bond, then a is none, R3 and R4 are none and, R1 and R2 are independently chosen from the group consisting of:
H,
ORa,
COORa,
NRaRb,
CONRaRb,
halogen,
NO₂,
CN,
SRa,
COSRa,
PRaRb,
O—P(O)(ORa)₂
P(O)(ORa)₂
(C₁-C₁₀)alkyl, which may be substituted by at least one possibly substituted (C₅-C₁₀)aryl or possibly substituted (C₅-C₁₀)heteroaryl,
(C₅-C₁₀)aryl or (C₅-C₁₀)heteroaryl which may be substituted by at least one possibly substituted (C₁-C₁₀)alkyl, and
functional groups being able to bind a vector, and functional groups having targeting properties,

or, R1 and R2 may form together with the carbon atoms carrying them a (C₅-C₁₀)cycloalkenyl, a (C₅-C₁₀)heterocycloalkenyl which may be substituted by at least one (C₁-C₁₀)alkyl and/or a CO group;
when b is a single bond, then a is a single bond and R1, R2, R3 and R4 are independently chosen from the group consisting of:
H,
ORa,
COORa,
(C₁-C₁₀)alkyl, which may be substituted by at least one possibly substituted (C₅-C₁₀)aryl or possibly substituted (C₅-C₁₀)heteroaryl,
(C₅-C₁₀)aryl or (C₅-C₁₀)heteroaryl which may be substituted by at least one possibly substituted (C₁-C₁₀)alkyl, and
functional groups being able to bind a vector, and functional groups having targeting properties;

R5 and R6 are independently chosen from the group consisting of:
(C₁-C₁₀)alkyl, which may be substituted by at least one possibly substituted (C₅-C₁₀)aryl or possibly substituted (C₅-C₁₀)heteroaryl,
(C₅-C₁₀)aryl or (C₅-C₁₀)heteroaryl, which may be substituted by at least one possibly substituted (C₁-C₁₀)alkyl;

wherein the (C₅-C₁₀)aryl, (C₅-C₁₀)heteroaryl and (C₁-C₁₀)alkyl groups of R5 and/or R6 are possibly substituted by at least one substituent chosen from the group consisting of:
ORa,
COH,
COORa,
NRaRb,
CONRaRb,
halogen,
NO₂,
CN,
SRa,
COSRa,
PRaRb,
O—P(O)(ORa)₂, and
P(O)(ORa)₂

wherein the (C₅-C₁₀)aryl, (C₅-C₁₀)heteroaryl, and (C₁-C₁₀)alkyl of R1, R2, R3, R4, R5 and R6 are possibly substituted by functional groups being able to bind a vector, and functional groups having targeting properties;

and wherein Ra and Rb are independently chosen among H, (C₅-C₁₀)aryl or (C₁-C₁₀)alkyl;

X is a heavy halogen chosen from the group consisting of: ¹²⁵I, ¹²³I, ¹²⁴I, ¹³¹I, and ²¹¹At;

L₁ and L₂ are independently chosen from the group consisting of:

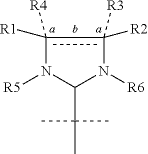

wherein R1, R2, R3, R4, R5, R6, and a, b are defined as above,
possibly substituted (C₅-C₁₀)heteroaryl,
CO group,
PRdReRf, in which Rd, Re and Rf are independently chosen from possibly substituted (C₁-C₁₀)alkyl and possibly substituted (C₅-C₁₀)aryl, and
possibly substituted monocyclic, polycyclic or acyclic (C₂-C₁₀)monoalkene,

or L₁ with L₂ may form together a possibly substituted monocyclic, polycyclic or acyclic (C₆-C₁₀)dialkene, and the pharmaceutically acceptable salts or bases thereof.

Surprisingly, the inventors found that complexes of formula (I), with rhodium in the (+I) oxidation state, are stabilized by N-heterocyclic carbenes ligands.

Moreover, rhodium in a (+I) oxidation state is a soft metallic cation which forms strong bonds with astatide.

### DEFINITIONS

The term “N-heterocyclic carbene” refers to a group of formula:

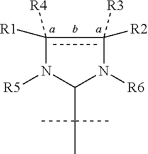

in which R1, R2, R3, R4, R5, R6, a and b are defined as above.

The term “(C₁-C₁₀)alkyl” means a saturated or unsaturated aliphatic hydrocarbon group which may be straight or branched having 1 to 10 carbon atoms in the chain. Preferred alkyl groups have 1 to 6 carbon atoms in the chain. “Branched” means that one or lower alkyl groups such as methyl, ethyl or propyl are attached to a linear alkyl chain. <<Lower alkyl>> means 1 to 4 carbon atoms in the chain which may be straight or branched.

By “alkene” or “alkenyl” is meant an unsaturated alkyl, comprising at least one double bond between two carbon atoms and comprising from 2 to 10 carbon atoms, preferably from 6 to 10 carbon atoms. A “monoalkene” refers to an alkene which has only one double bond, a dialkene refers to an alkene which has two double bonds. Alkene groups can be acylic, monocyclic or polycyclic, for example bicyclic, or acyclic.

According to the invention, “(C₅-C₁₀)cycloalkenyl” refers to a cyclic alkenyl group comprising between 5 to 10 carbon atoms.

The term “(C₅-C₁₀)heterocycloalkenyl” refers to a cyclic alkenyl group comprising 5 to 10 carbon atoms and wherein one or more carbon atom(s) are replaced by one or more heteroatom(s) such as nitrogen atom(s), oxygen atom(s) and sulphur atom(s); for example 1 or 2 nitrogen atom(s), 1 or 2 oxygen atom(s), 1 or 2 sulphur atom(s) or a combination of different heteroatoms. Examples of heterocycloalkenyl moieties include, but are not limited to pyrimidine or hexahydropyrimidine.

By “alkynyl” is meant an unsaturated alkyl, comprising at least one triple bond between two carbon atoms and comprising from 2 to 10 carbon atoms, preferably from 6 to 10 carbon atoms.

The term “(C₅-C₁₀)aryl” refers to an aromatic monocyclic, bicyclic, or tricyclic hydrocarbon ring system, wherein any ring atom capable of substitution may be substituted by a substituent. Examples of aryl moieties include, but are not limited to, phenyl and naphthyl.

The term “(C₅-C₁₀)heteroaryl” refers to an aromatic monocyclic, bicyclic, or tricyclic hydrocarbon ring system, wherein any ring atom capable of substitution may be substituted by a substituent and wherein one or more carbon atom(s) are replaced by one or more heteroatom(s) such as nitrogen atom(s), oxygen atom(s) and sulphur atom(s); for example 1 or 2 nitrogen atom(s), 1 or 2 oxygen atom(s), 1 or 2 sulphur atom(s) or a combination of different heteroatoms. Examples of heteroaryl moieties include, but are not limited to, imidazole.

The term “halogen” (or “Hal”) refers to the atoms of the group 17 of the periodic table (halogens) and includes in particular fluorine, chlorine, bromine, and iodine atom.

The term “heavy halogen” refers to iodine and astatine.

The expression “CO” refers to:
a group in which the carbon atom belongs to the (C₅-C₁₀)cycloalkenyl or (C₅-C₁₀)heterocycloalkenyl group formed by R1 and R2 with the carbon atoms carrying them, or
a CO group linked to the rhodium atom when L₁ and/or L₂ is CO.

The term “vector” refers to a molecule being able to recognize a biological target tissue (depending on the pathology to be treated or detected).

In particular, this term refers to biomolecules.

The term “vector” may also refer to organic compounds binding cells or organic compounds transported by transporters expressed by cells (e.g., but not limited to, glucose, amino-acids, biogenic amines), peptides binding specific receptors (e.g. but not limited to somatostatine, cholecystokinine, neurotensine receptors), haptens and drugs.

The term “vector” may also refer to a nanocarrier compound able to recognize the target cells such as a nanocapsule, a liposome, a dendrimer or a carbon nanotube. These nanocarriers may be linked if necessary to tumor specific ligands.

By “biomolecules”, it is understood an antibody or fragments thereof or any antibody construct (like minibodies, diabodies etc. . . . resulting from antibody engineering) as well as recombinant proteins or synthetic peptides selected to bind target cells (e.g., but not limited to, affibodies)).

The expression “functional groups being able to bind a vector” refers to a chemical group which is reactive towards the chemical functions of a vector and thus allows the formation of a stable chemical bond between the vector and the synthon (which is the compound of formula (I)).

The expression “functional group having targeting properties” refers to a chemical group being a vector (e.g., but not limited to peptides, haptens and drugs). The expression “functional group having targeting properties” may also refer to a chemical group which gives the properties of a vector to a compound of formula (I). In particular, this term may refer, but are not limited to haptens, peptides or drugs.

Among such functional groups being able to bind a vector or having targeting properties, the followings may be cited:
NO₂,

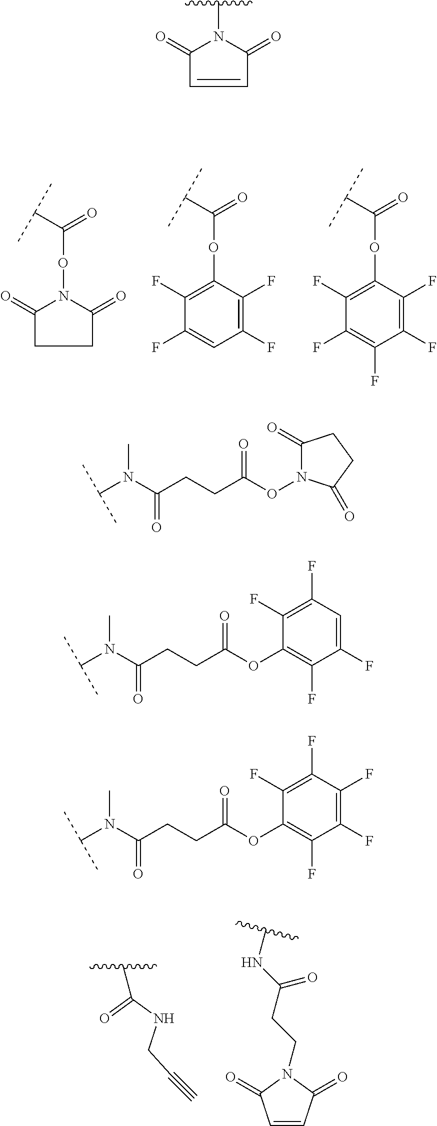

OH,
COH,
COOH,
NH₂,
NHRk wherein Rk is chosen from the group of possibly substituted (C₁-C₁₀)alkyl and possibly substituted (C₅-C₁₀)aryl,

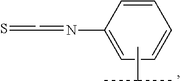

NCS,
NCO,
SH,

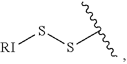

with Rl being (C₁-C₁₀)alkyl, (C₅-C₁₀)aryl or (C₅-C₁₀)heteroaryl, such as

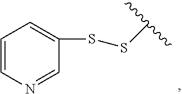 [SMILES: *SSc1cccnc1]

(C₂-C₁₀)alkynyl,
(C₁-C₁₀)alkylene-N₃,
N₃ group, and
biotinyl and its derivatives.

In a preferred embodiment, the functional groups are chosen in the group consisting of:

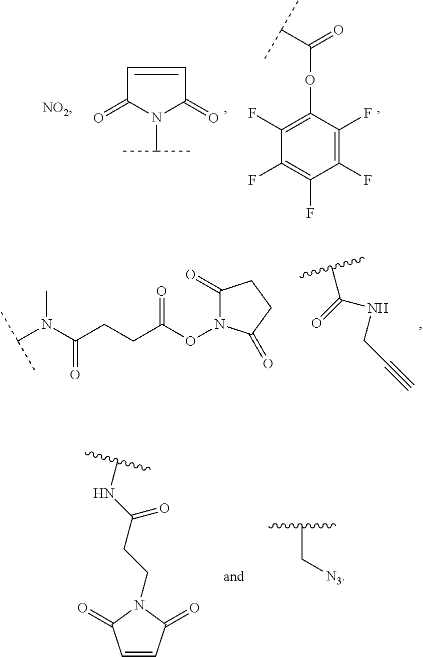

In a particular embodiment, the present invention relates to a compound having the formula (I-1):

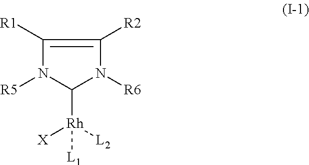

in which R1, R2, R5, R6, L1, L2 and X are defined as above.

In a preferred embodiment, R1 and R2 are chosen among:
H,
NO₂,
a substituted (C₁-C₁₀)alkyl,

or, R1 and R2 may form together with the carbon atoms carrying them a (C₅-C₁₀)cycloalkenyl, a (C₅-C₁₀)heterocycloalkenyl possibly substituted by at least one (C₁-C₁₀)alkyl and/or a CO group.

In another preferred embodiment, R1 and R2 are chosen among:
H,
NO₂,
ethyl group substituted by functional group being able to bind a vector, and functional groups having targeting properties,

or R1 and R2 may together form a hexahydropyrimidine substituted by one or more (C₁-C₁₀)alkyl and/or a CO group.

Preferably, R1 and R2 are H or NO₂.

Preferably, R1 and R2 are H.

In particular embodiment, R5 is a (C₁-C₁₀)alkyl substituted by a possibly substituted (C₅-C₁₀)aryl.

In a particular embodiment, R5 is

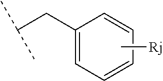

in which Rj is chosen from the group consisting of H, functional groups being able to bind a vector, and functional groups having targeting properties, such as:
NO₂,

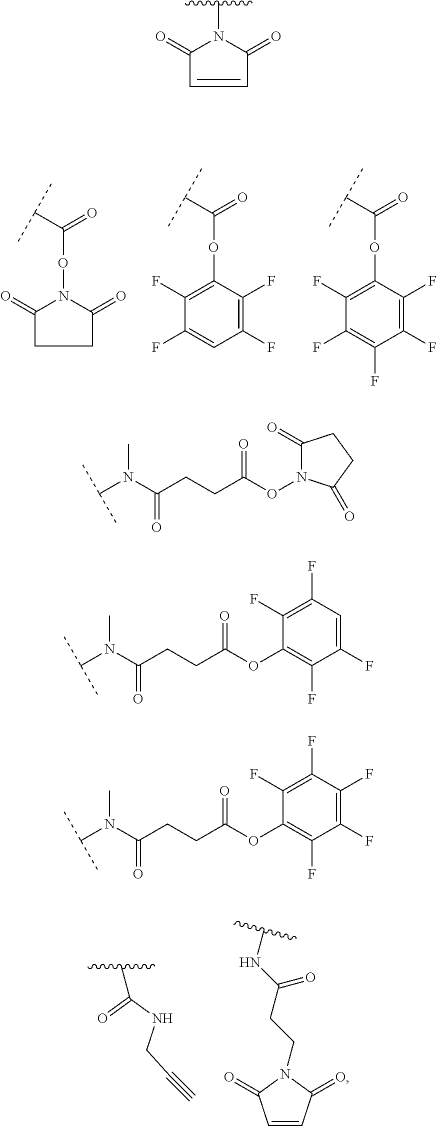

OH,
COH,
COOH,
NH₂,
NHRk wherein Rk is chosen from the group of possibly substituted (C₁-C₁₀)alkyl and possibly substituted (C₅-C₁₀)aryl,

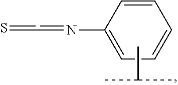

NCS,
NCO,
SH,

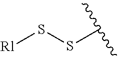

with Rl being (C₁-C₁₀)alkyl, (C₅-C₁₀)aryl or (C₅-C₁₀)heteroaryl, such as

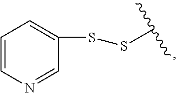 [SMILES: *SSc1cccnc1]

(C₂-C₁₀)alkynyl,
(C₁-C₁₀)alkylene-N₃,
N₃ group, and
biotinyl and its derivatives.

In a preferred embodiment, the functional groups are chosen in the group consisting of:

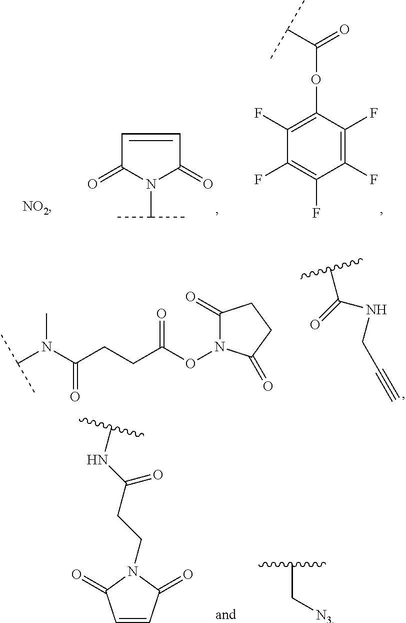

In another particular embodiment, R5 is a benzyl group.

Preferably, R6 is chosen among a preferably substituted (C₅-C₁₀)aryl, a (C₅-C₁₀)heteroaryl and a (C₁-C₁₀)alkyl.

In a particular embodiment, R6 is

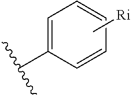

in which Ri is chosen from the group consisting of H, NO₂, and (C₁-C₁₀)alkyl.

In another embodiment, R6 is a methyl group.

In another embodiment, R6 is a 4-nitrophenyl group.

According to the invention, L₁ and L₂ are chosen among 2-electrons donating ligands, classified as L according to the Covalent Bond Classification (Green, M. L. H. (1995-09-20). “A new approach to the formal classification of covalent compounds of the elements”. Journal of Organometallic Chemistry 500 (1-2): 127-148.) These ligands play a role in the stabilisation of the compounds of the invention.

In a particular embodiment, L₁ and L₂ may form together a possibly substituted monocyclic, polycyclic or acyclic (C₆-C₁₀)dialkene.

Preferably, L₁ together with L₂ form a 1,5-cyclooctadiene or a norbornadiene.

More preferably, L₁ together with L₂ form a 1,5-cyclooctadiene.

In a particular embodiment, X is ²¹¹At.

In another embodiment, X is a heavy halogen chosen among the group consisting of ¹³¹I, ¹²⁴I or ¹²⁵I.

In another embodiment, the compound according to the invention comprises at least one of R1, R2, R3, R4, R5 and R6 comprising a functional group being able to bind a vector.

In a particular embodiment, the compound of the invention comprises at least one of R1, R2, R3 and R4 which is a functional group being able to bind a vector, and/or at least one of the (C₅-C₁₀)aryl, (C₅-C₁₀)heteroaryl, and (C₁-C₁₀)alkyl of R1, R2, R3, R4, R5 and R6 substituted by at least one functional group being able to bind a vector.

Some specific compounds of the invention have one of the following formulae:

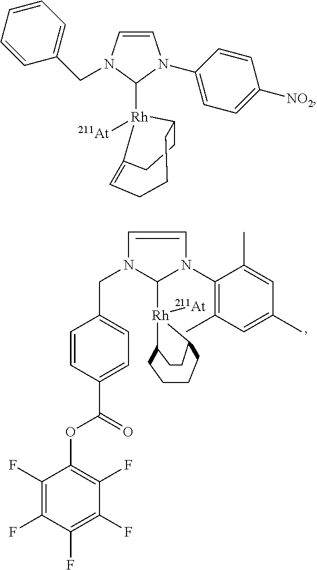

The invention also relates to compounds, having the formula (II):

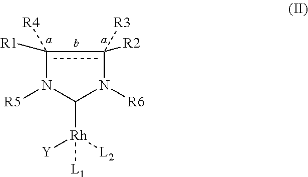

wherein a, b, R1, R2, R3, R4, R5, R6, L₁ and L₂ are as defined as above and Y is a halogen atom.

In a particular embodiment, Y is Cl.

In another embodiment, Y is Br or I.

Methods of Preparation:

The present invention also relates to a method for the preparation of a compound having formula (I) defined as above, comprising a step of radiolabelling of a compound of formula (II) defined as above.

In a particular embodiment, the step of radiolabelling comprises the ligand substitution of Y in a compound of formula (II) as defined above, with X at the (—I) oxidation state.

According to another embodiment, the ligand substitution reaction time is comprised from 45 to 60 minutes, and is preferably 60 minutes at 37° C. (see Example 10).

In a particular embodiment, the ligand substitution reaction time is comprised from 10 to 60 minutes, and is preferably 15 minutes at 60° C.

In a particular embodiment, the ligand substitution reaction time is of 5 minutes at 100° C.

In a particular embodiment, the radiolabelling comprises the reaction of a reducing agent (e.g., but not limited to, cystein) with ²¹¹At.

In a particular embodiment, an aqueous solution of cystein may be added to astatine in DIPE (diisopropylether) which may contain nitric acid at 3M. The resultant mixture may form a biphasic system in a ratio (DIPE+Nitric acid)/Water of 10/8.

The compounds of formula (II) or (I-2) can be obtained by preparation methods known to a man skilled in art.

Particular methods of preparation are described below without limitation of the invention.

In a particular embodiment, the compounds of formula (II) as defined above are obtained by a process comprising the following steps:
the reaction of a compound of formula (III)

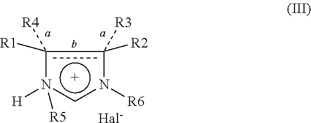

wherein a, b, R1, R2, R3, R4, R5, R6 are defined as above, and Hal represents a halogen atom, with Ag₂O,
followed by the reaction of the resulting mixture with a compound [Rh(L1 L2)Y]₂, wherein L1, L2 and Y are as defined above.

According to another aspect, a method for the preparation of a compound of formula (I-2)

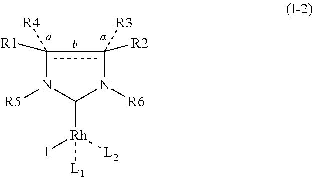

wherein a, b, R1, R2, R3, R4, R5, R6, L₁ and L₂ are defined as above, comprises the following steps:
the reaction of a compound of formula (III):

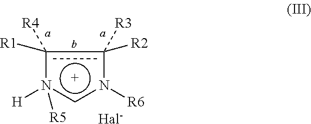

wherein a, b, R1, R2, R3, R4, R5, R6 are defined as above, and Hal representing a halogen atom, with Ag₂O,
followed by the reaction of the resulting mixture with a compound [Rh(L1L2)Y]₂, Y being as above, in the presence of KI.

In a particular embodiment, Hal is CI, Br or I.

In a particular embodiment, the preparation of a compound of formula (I)

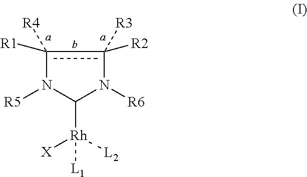

wherein a, b, R1, R2, R3, R4, R5, R6, L₁, L₂ and X are defined as above comprises the following steps:
   the reaction of a compound of formula (III):

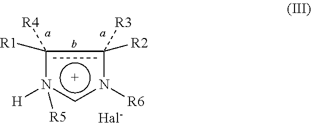

wherein a, b, R1, R2, R3, R4, R5, R6 are as defined above, and Hal representing a halogen atom, with Ag₂O,
followed by the reaction of the resulting mixture with a compound [Rh(L1L2)Y]₂, Y being as defined above, in the presence of X at the (—I) oxidation state.

In a particular embodiment, Hal is CI, Br or I.

In a particular embodiment, the [Rh(L1 L2)Y]₂ compound is represented by a 1,5-cyclooctadiene rhodium chloride dimer, which is a chemical compound with the formula Rh₂Cl₂(C₈H₁₂)₂, commonly abbreviated [RhCl(COD)]₂. It may be represented according to the following formula:

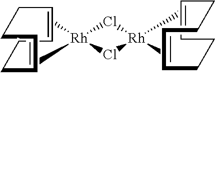

The abbreviation COD refers to a cyclooctadiene group in the whole application.

Radiolabelling Methods:

In a particular embodiment, the present invention relates to the radiolabelling of a compound of formula (II) using ligand substitution to form a compound of formula (I) bearing a functional group having targeting properties.

In another embodiment, the present invention relates to the radiolabelling of a compound of formula (II) using ligand substitution to form a compound of formula (I) bearing functional groups being able to bind a vector in a first step and coupling the compound of formula (I) to a vector (e.g., but not limited to, antibodies, fragments of antibodies and their derivatives) in a second step.

In still another embodiment, the present invention relates to the coupling of a compound of formula (II) bearing functional groups being able to bind a vector to a vector (e.g., but not limited to, antibodies, fragments of antibodies and their derivatives) in a first step and then to the radiolabelling of the resulting compound using ligand substitution to form a compound of formula (I) linked to a vector. Preferably, the vector is an antibody.

The methods of coupling are well known by a man skilled in the art and may be found in the following references:
David M. Wong and Shan S. Jameson, Chemistry of Protein and Nucleic Acid Cross-Linking and Conjugation Second Edition, CRC Press 2011 (NY), 604 p.;
N. L. Benoiton, Peptide Bond Formation: Active Esters in HoubenWeyl Methods of Organic Chemistry Synthesis of Peptides and Peptidomimetics (2001), WORKBENCH EDITION;
Emmanuel Basle, Nicolas Joubert and Mathieu Pucheault, Protein Chemical Modification on Endogenous Amino Acids, Chemistry & Biology (2010), Volume 17, Issue 3, 213-227;
Sletten, E. M.; Bertozzi, C. R. Bioorthogonal Chemistry: Fishing for Selectivity in a Sea of Functionality. Angew. Chem. Int. Ed. (2009), 48, 6974-6998;
Shuang Liu, Bifunctional Coupling Agents for Radiolabeling of Biomolecules and Target-Specific Delivery of Metallic Radionuclides, Advanced Drug Delivery Reviews, Volume 60, Issue 12, 15 Sep. 2008, Pages 1347-1370;
Wu A M, Senter P D, Arming antibodies: prospects and challenges for immunoconjugates. Nat Biotechnol 2005, 23:1137-1146; and
Alan R. Fritzberg, Ronald W. Berninger, Stephen W. Hadley and Dennis W. Wester, Approaches to Radiolabeling of Antibodies for Diagnosis and Therapy of Cancer, Pharmaceutical Research (1988), Volume 5, Number 6, 325-334

These methods are also known as conjugation methods wherein the compound of formula (I), linked to the vector through the functional group of said compound, is called “a conjugate”.

The present invention relates to a conjugate comprising at least one compound according to the invention, covalently linked to a vector through the functional group of said compound.

Pharmaceutical Compositions:

The present invention also relates to a pharmaceutical composition, comprising a compound having formula (I) as defined above, in association with at least one pharmaceutically acceptable excipient, said compound being if necessary coupled to a vector preferably chosen from biomolecules, more preferably antibodies, and nanocarrier compounds.

The present invention also relates to a drug, comprising a compound having formula (I) as defined above, in association with at least one pharmaceutically acceptable excipient, said compound being if necessary coupled to a vector preferably chosen from biomolecules such as antibodies and nanocarrier compounds.

While it is possible for the compounds of the invention having formula (I) to be administered alone, it is preferred to present them as pharmaceutical compositions. The pharmaceutical compositions, both for veterinary and for human use, useful according to the present invention comprise at least one compound having formula (I) as above defined, together with one or more pharmaceutically acceptable carriers and possibly other therapeutic ingredients.

In certain preferred embodiments, active ingredients necessary in combination therapy may be combined in a single pharmaceutical composition for simultaneous administration.

As used herein, the term “pharmaceutically acceptable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like.

The preparation of a pharmacological composition that contains active ingredients dissolved or dispersed therein is well understood in the art and need not be limited based on formulation. Typically such compositions are prepared as injectables either as liquid solutions or suspensions; however, solid forms suitable for solution, or suspensions, in liquid prior to use can also be prepared. The preparation can also be emulsified. In particular, the pharmaceutical compositions may be formulated in solid dosage form, for example capsules, tablets, pills, powders, dragees or granules.

The choice of vehicle and the content of active substance in the vehicle are generally determined in accordance with the solubility and chemical properties of the active compound, the particular mode of administration and the provisions to be observed in pharmaceutical practice. For example, excipients such as lactose, sodium citrate, calcium carbonate, dicalcium phosphate and disintegrating agents such as starch, alginic acids and certain complex silicates combined with lubricants such as magnesium stearate, sodium lauryl sulphate and talc may be used for preparing tablets. To prepare a capsule, it is advantageous to use lactose and high molecular weight polyethylene glycols. When aqueous suspensions are used they can contain emulsifying agents or agents which facilitate suspension. Diluents such as sucrose, ethanol, polyethylene glycol, propylene glycol, glycerol and chloroform or mixtures thereof may also be used.

The pharmaceutical compositions can be administered in a suitable formulation to humans and animals by topical or systemic administration, including oral, rectal, nasal, buccal, ocular, sublingual, transdermal, rectal, topical, vaginal, parenteral (including subcutaneous, intra-arterial, intramuscular, intravenous, intradermal, intrathecal and epidural), intracisternal and intraperitoneal. It will be appreciated that the preferred route may vary with for example the condition of the recipient.

The formulations can be prepared in unit dosage form by any of the methods well known in the art of pharmacy. Such methods include the step of bringing into association the active ingredient with the carrier which constitutes one or more accessory ingredients.

In general the formulations are prepared by uniformly and intimately bringing into association the active ingredient with liquid carriers or finely divided solid carriers or both, and then, if necessary, shaping the product.

Pharmaceutical Uses:

The compounds having formula (I) as defined above may be used alone in pharmaceutical compositions or may be coupled to a vector before their administration. The term “vector” is defined above and refers in particular to a biomolecule such as antibodies or fragments thereof or any antibody construct (like minibodies, diabodies etc. . . . resulting of antibody engineering), peptides or haptens, or to a nanocarrier compound able to recognize the target cells such as a nanocapsule, a liposome, a dendrimer or a carbon nanotube. Said target cells are the cells in which the radionuclides have to be transported in order to kill or detect said cells.

The present invention also relates to a compound having formula (I) as defined above (administered alone or coupled to a vector), for its use in the treatment or localization of tumors.

In particular, the present invention also relates to a compound having formula (I) as defined above wherein X is ²¹At (administered alone or coupled to a vector), for its use in the treatment of tumors.

According to an advantageous embodiment, the present invention relates to a compound having formula (I) as defined above wherein X is ¹²³I or ¹²⁴I (administered alone or coupled to a vector), for its use in the detection of tumors.

According to an advantageous embodiment, the present invention relates to a compound having formula (I) as defined above wherein X is ¹³¹I or ²¹¹At (administered alone or coupled to a vector), for its use in the treatment of tumors.

According to a particular embodiment, the present invention relates to a compound having formula (I) as defined above in the treatment of small size primary tumor diseases, metastatic cancer diseases, preferably micrometastasis, and disseminated hematological cancers.

The term “small size primary tumor diseases” refers to primary tumors which have a diameter of 5 cm maximum.

The terms “metastasis” or “metastatic cancer diseases” refer to secondary tumors that are formed by cells from a primary tumor which have moved to another localization. Metastasis are called “micrometastasis” or “micrometastatic diseases” when their size does not exceed 1 cm diameter.

The term “hematological cancers” refers to types of cancer that affect blood, bone marrow, and lymph nodes such as myelomas, lymphomas or leukemias.

In a particular embodiment, the present invention refers to a compound of formula (I) as defined above for its use in the treatment of a disease chosen from the group consisting of myeloma, lymphoma, acute myeloid or lymphoblastic leukemia, prostate bone metastasis, disseminated peritoneal ovarian metastasis, glioma, small cells lung cancer, and CEA positive tumors metastasis (liver, colonic carcinoma, medullary thyroid cancer, small cell lung cancer). By “CEA positive liver metastasis” is meant a tumor metastasis which is positive to Carcino Embryonic Antigen.

In a particular embodiment, the invention relates to a method of treatment of tumors comprising the administration to a patient of a compound having formula (I) as defined above (administered alone or coupled to a vector).

In a particular embodiment, the invention also relates to a method of localisation of tumors comprising the administration to a patient of a compound having formula (I) as defined above (administered alone or coupled to a vector).

Some examples of methods preparation are given below, without limitation of the present invention.

### EXAMPLES

The materials and methods used in the examples are described below:

Chemicals purchased from commercial sources were reagent grade or better and were used without further purification. All solvents were obtained as HPLC grade or better. Nitric acid in analytical grade and sodium sulphite were obtained from Merck. Diisopropyl ether was obtained from Carlo Erba Reagents in analytical grade. Solvents for HPLC analysis were obtained in HPLC grade and degassed before use by ultrasonication for 20 minutes.

Astatine-211 was produced using the ²⁰⁹Bi(α,2n)²¹¹At reaction by bombarding a 240 μm thick natural bismuth layer on copper target with a 1.95-2.15 μA particle beam of 28 MeV α-particle during 2 hours.

The following protocol was used to produce astatine solution in DIPE (0.1 MBq/μl). The irradiated target was placed in a beaker and 500 μL of 65% nitric acid was added drop-wise on the bismuth layer. After 10 min, the acidic solution was withdrawn and evaporated to dryness at 165° C. The acidic target attack/evaporation to dryness steps were repeated four times. After cooling, the residue was dissolved in 3 mL of 32% nitric acid and astatine-211 was then extracted in DIPE (500 μL). The harvested activity was determined in an ACAD 2000 ionization chamber.

Spectral Analysis:

¹H and ¹³C{H} NMR spectra were obtained on a Bruker AC (400 MHz). Proton chemical shifts are expressed as parts per million (ppm) using solvent peak as internal reference. Mass spectral data were obtained on a Bruker Daltonics Esquire HCT Ultra Ion Trap using electrospray ionisation.

Analytical Chromatography:

Silica gel chromatography was conducted with 60-430 μm 60 Å silica gel (Carlo Erba SDS). TLC was carried out on precoated silica gel 60 F254 TLC plastic sheets (Merck) and reactions were monitored by revelation under UV light (254 nm). A gradient elution of heptane and ethyl acetate was used.

HPLC analysis was performed using a Waters HPLC System equipped with a Waters 486 Tuneable Absorbance Detector and a Packard Bioscience Flow Scintillation Analyser 150 TR. For iodine-125 readiolabelling, fractions of 1 ml each were collected and counted. A gradient elution of heptane (A) and ethyl acetate (B) (0-3 min: A, 3.01-9.5 min.: 70/30 (A/B), 9.51-15 min.: 60/40 (A/B), 15.01-20 min.: A) at 2 ml/min. A Waters Prep NovaPack HR Silica column was used for all rhodium complexes analytic controls. Radio-TLC was carried out on precoated silica gel 60 F₂₅₄ TLC plastic sheets (Merck) eluted with heptane/acetone 3:2 or dichloromethane. Radio-TLC plates were examined using a Typhoon 9410 Variable Mode Imager (GE Healthcare Bioscience). Size exclusion chromatographies were performed on NAP5 cartridges eluted with phosphate buffered saline solution. Purification by silica gel chromatography was performed with SepPack silica gel cartridge (Oasis, France).

### Example 1

### Preparation of a compound of formula (III) 1-benzyl-3-(4-nitrophenyl)imidazolium bromide

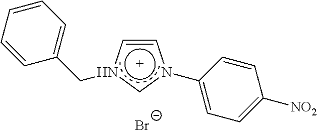

Benzyl bromide (712 μL, 6 mmol) and 4-nitrophenylimidazole (222 mg, 1.2 mmol.) were stirred in 10 ml of anhydrous THF under reflux for 45 h and under inert atmosphere. The precipitate was then filtered, washed with THF and dried under reduced pressure to give 393 mg (91%) of 1-benzyl-3-(4-nitrophenyl)imidazolium bromide (Example 1) as an off white powder.

¹H NMR (DMSO-d₆, 400.13 MHz): δ 10.37 (t, 1H, J=1.5 Hz, N—CH—N), 8.57 (1H, m, H(imidazolium backbone)), 8.55 (2H, m, H(nitrobenzyl)), 8.19 (1H, m, H(imidazolium backbone)), 8.17 (2H, m, H(nitrophenyl)), 7.64-7.58 (2H, m, Hₘ(benzyl)), 7.52-7.43 (3H, m, Hₚ+Hₒ(benzyl)), 5.61 (2H, s, CH₂).

¹³C NMR (DMSO-d₆, 100.16 MHz): δ 147.7 (carbene), 139.5, 134.4, 129.1, 129.0, 128.9, 125.7, 123.7, 123.2, 121.8, 52.7 (CH₂).

MS (ES⁺) calcd for (M-Bn-FH)⁺190.1 (100%), 191.1 (11%) (M-Br)⁺: 280.1 (100%), 281.1 (18.7%), (2M-Br)⁺: 639.1 (100%), 641.1 (95%), 640.1 (36%), 642.1 (36%) Found (M-Bn-FH)⁺190.0 (100%) (M-Br)⁺: 280.1 (100%), 280.8 (18.9%), (2M-Br)⁺: 639.0 (95%), 641.0 (100%)

### Example 2

### Preparation of a compound of formula (II) (1-benzyl-3-(4-nitrophenyl)imidazolidene)(cyclooctadiene) rhodium (+I) chloride

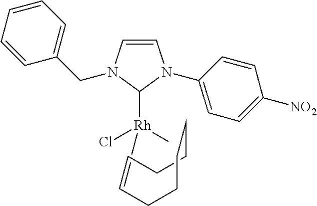

1-benzyl-3-(4-nitrophenyl)imidazolium bromide (Example 1) (44 mg, 0.122 mmol) and silver oxide (14 mg, 0.061 mmol.) were stirred in 4 ml of degassed methylene chloride at room temperature for 2 h in the dark. The resultant mixture was filtered over a celite pad. [Rh(COD)Cl]₂ (30 mg., 0.061 mmol.) was then added. The solution was stirred 18 h at room temperature. Volatiles were removed under reduced pressure and the residue was purified by silica gel chromatography (99.5/0.5 DCM/MeOH). After concentration, heptane was added until precipitates appeared. The product was recovered by filtration and dried under reduced pressure. 44 mg (65%) of (1-benzyl-3-(4-nitrophenyl)imidazolidene)(cyclooctadiene) rhodium (+I) chloride (Example 2) was then obtained as dark yellow powder.

¹H NMR (CDCl₃, 400.13 MHz): δ 8.59 (4H, m, H(nitrobenzyl)), 7.52-7.35 (5H, m, H(benzyl)), 7.21 (1H, d, J=2.0 Hz, H(imidazolidene backbone)), 6.91 (1H, d, J=2.0 Hz, H(imidazolidene backbone)), 5.99 (2H, m, CH₂), 5.31-5.12 (1H, m, CH(COD)), 5.12-5.03 (1H, m, CH(COD)), 3.23-3.14 (1H, m, CH(COD)), 2.58-2.50 (1H, m, CH(COD)), 2.40-2.28 (1H, m, CH₂(COD)), 2.25-2.08 (2H, m, CH₂(COD)), 1.93-1.73 (3H, m, CH₂(COD)), 1.66-1.54 (2H, m, CH₂(COD))

¹³ NMR (CDCl₃, 100.16 MHz): δcarbene signal not found, 146.9, 145.1, 135.6, 129.1 (CH(benzyl)), 128.6 (CH(benzyl)), 128.5 (CH(benzyl)), 125.1 (CH(nitrophenyl)), 124.3 (CH(nitrophenyl)), 122.2 (CH(imidazolidene)), 121.0 (CH(imidazolidene)), 99.5 (d, J_{C-Rh}=7.0 Hz, CH(COD)), 98.7 (d, J_{C-Rh}=7.0 Hz, CH(COD), 69.3 (d, J_{C-Rh}=14.0 Hz, CH(COD), 68.8 (d, J_{C-Rh}=14.0 Hz, CH(COD), 55.6 (CH₂), 32.7 (CH₂(COD)), 32.3 (CH₂(COD)), 28.6 (br, m, CH₂(COD)), 28.0 (CH₂(COD)).

LRMS (ES⁺) calcd for (M-Cl)⁺: 490.1 (100%), 491.1 (27%), (2M-2Cl+CN)⁺: 1006.2 (100%), 1007.2 (53%), 1008.2 (14%) (2M-Cl)⁺. 1015.2 (100%), 1016.2 (54%), 1017.2 (34%), 1018.2 (17%) Found: (M-Cl)⁺: 490.3 (100%), 491.1 (30%) (2M-2Cl+CN)⁺: 1006.4 (100%), 1007.2 (54%), 1008.1 (15%) (2M-Cl)⁺. 1015.3 (100%), 1016.2 (51%), 1017.2 (41%), 1018.1 (21%)

HPLC: t_{R}=11.7 min.

### Example 3

### Preparation of a compound of formula (I) (¹²⁵I)(1-benzyl-3-(4-nitrophenyl)imidazolidene)(cyclooctadiene)rhodium(+1)

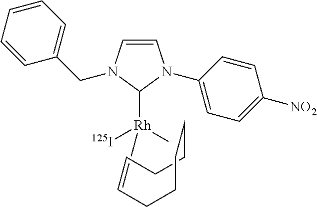

To 16 μl of aqueous solution of (¹²⁵I)NaI (0.12-3.7 MBq) were added 40 μl of the compound Example 2 in ACN (21.5 μmol/ml). After 30 s. of vigorous stirring, the mixture was incubated at 100° C. during 5 min.

HPLC t_{R}=8 min.

TLC: R_{f}=0.45

### Example 4

### Preparation of a compound of formula (I) (²¹¹At)(1-benzyl-3-(4-nitrophenyl)imidazolidene)(cyclooctadiene) rhodium(+I)

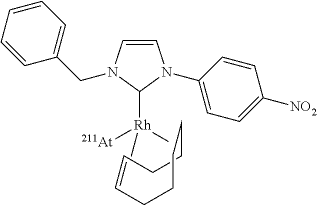

To 10 μL of astatine in DIPE (0.75-1.05 MBq) was added cysteine (7.1 mg/ml in water). After 30 s. of vigorous stirring, the compound of Example 2 in ACN was added. The mixture was stirred and incubated at r.t., 37, 60 or 100° C.

Optimized procedure To 10 μL of astatine in DIPE (0.75-1.05 MBq) were added 8 μL of cysteine (7.1 mg/ml in water). After 30 s. of vigorous stirring, 40 μL of Example 2 in ACN (3 mg/ml) were added. The mixture was stirred and incubated 15 min at 60° C. or 5 min. at 100° C.

TLC: R_{f}=0.52

HPLC: t_{R}=6.8 min.

### Example 5

### Preparation of 1-mesityl-1H-imidazole

(Compound Described in Synthesis 2003 (17), 2661-2666)

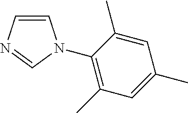

2,4,6-Trimethylaniline (12.2 ml, 87 mmol.) and 40% aq. glyoxal (10 ml, 87 mmol) were stirred 16 h at room temperature in 25 ml of methanol. Ammonium chloride (9.3 g, 174 mmol.), paraformaldehyde (5.2 g, 174 mmol) were then added. After adding 200 ml of methanol, the mixture was stirred under reflux for 1 h. 12 ml of orthophosphoric acid was then gently added over a period of 10 min. The resulting mixture was then stirred under reflux for 8 h. Volatile materials were removed under reduced pressure then the residue was poured onto ice and neutralized with aq. 40% KOH until pH 9. The resulting mixture was extracted using ethyl acetate (4×100 ml). Organic phases were combined, washed with distillated water, brine and dried with Na2So4. After filtration, volatiles were removed and the crude product was chromatographied on silica gel (Hept/AcOEt (50:50)). After concentration, the product was recristallised using diethyl ether. Filtration followed by washing with diethyl ether afforded the 1-mesityl-1H-imidazole as an off-white product (4.03 g, 25%).

¹H NMR (CDCl₃, 400.13 MHz): 7.48 (t, J=1.2 Hz, N—CH—N, 1H), 7.25 (t, J=1.2 Hz, N—CH—CH, 1H), 6.98 (s, Hmesityl, 2H), 6.90 (t, J=12 Hz, N—CH—CH, 1H), 2.34 (s, CH₃, 3H), 1.99 (s, CH₃, 3H).

### Example 6

### Preparation of perfluorophenyl 4-(bromomethyl)benzoate

(Compound Described in Eur. J. Inorg. Chem. 2008, 3359-3366)

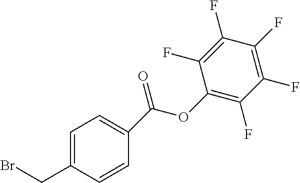 [SMILES: O=C(Oc1c(F)c(F)c(F)c(F)c1F)c1ccc(CBr)cc1]

To 4-(bromomethyl)benzoic acid (430 mg, 2 mmol) and pentafluorophenol (368 mg, 2 mmol) in 6.2 ml of AcOEt/DMF (60/2) was added N,N′-dicyclohexylcarbodiimide (413 mg, 2 mmol) at 0° C. under inert atmosphere. The mixture was stirred at 0° C. during 1 h then 2 h at room temperature. The solution was filtered off then volatiles were removed under reduced pressure to give perfluorophenyl 4-(bromomethyl)benzoate as a white solid (372 mg, 50%).

¹H NMR (DMSO-d6, 400.13 MHz): 8.22 (d, J=8.4 Hz, Ho, 2H), 7.77 (d, J=8.4 Hz, Hm, 2H), 4.87 (s, CH₂, 2H)

¹⁹F NMR (DMSO-d6, 376.50 MHz): −154.4-−154.5 (m, 2F, Far), −158.5 (t, J=22.7 Hz, 1F, Far), −163.3-−163.4 (m, 2F, Far).

### Example 7

### Preparation of a compound of formula (III) 3-mesityl-1-(4-((perfluorophenoxy)carbonyl)benzyl)-1H-imidazolium bromide

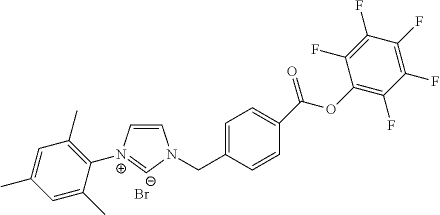 [SMILES: Cc1cc(C)c(-[n+]2ccn(Cc3ccc(C(=O)Oc4c(F)c(F)c(F)c(F)c4F)cc3)c2)c(C)c1 ; [Br-]]

To the compound of Example 5 (93 mg, 0.5 mmol) was added the compound of Example 6 (190 mg, 0.5 mmol) in 3 ml of anhydrous THF under argon. The mixture was refluxed overnight. After cooling at room temperature, the mixture was filtered. The precipitate was washed with cold THF then dried under vacuum to give 3-mesityl-1-(4-((perfluorophenoxy)carbonyl)benzyl)-1H-imidazolium bromide as a white solid (150 mg, 53%).

¹H NMR (DMSO-d6, 400.13 MHz): 9.68 (s, N—CH—N, 1H), 8.32 (d, J=8.4 Hz, Ho(benzyl), 2H), 8.19-8.18 (m, H(imidazolium backbone), 1H), 8.06 (m, H(imidazolium backbone), 1H), 7.76 (d, J=8.4 Hz, Hm (benzyl), 2H), 7.02 (s, H(mesityl), 2H), 5.77 (s, CH₂, 2H), 2.37 (s, CH₃, 3H), 2.08 (s, CH₃, 3H)

¹³C NMR (DMSO-d6, 100.16 MHz): 162.0, 142.2, 140.5, 138.2, 134.4, 131.4, 131.3, 129.4, 129.3, 126.3, 124.6, 123.6, 52.0, 20.7, 17.1.

¹⁹F NMR (DMSO-d6, 376.50 MHz): −154.5-−154.6 (m, 2F, Far), −158.3 (t, J=23.1 Hz, 1F, Far), −163.2-−163.3 (m, 2F, Far).

### Example 8

### Preparation of a compound of formula (II) (3-mesityl-1-(4-((perfluorophenoxy)carbonyl)benzyl)imidazolidene)(cyclooctadiene) rhodium (+I) chloride

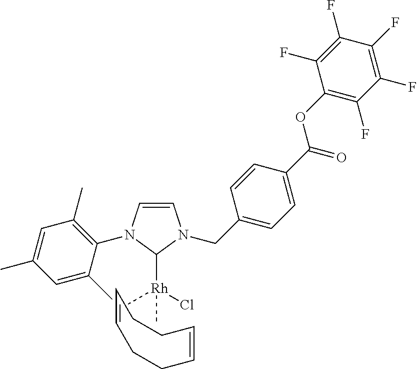

To one bead of molecular sieves (4A), the compound of Example 7 (44 mg, 0.077 mmol) and Ag₂O (14 mg, 0.06 mmol) was added 3 ml of degassed dichloromethane. The mixture was stirred in the dark at room temperature for 3 h. [Rh(COD)Cl]₂ (30 mg, 0.06 mmol) in 1 ml of dichloromethane was added and the solution was stirred overnight at room temperature under argon. The solution was filtered over Celite and concentrated under vacuo. The crude product was precipitated using Heptane/Dichloromethane (10/2) and after filtration and washing with heptane, the (3-mesityl-1-(4-((perfluorophenoxy)carbonyl)benzyl)imidazolidene) (cyclooctadiene) rhodium (+1) chloride was obtained as a yellow powder (50 mg, 85%).

¹H NMR (DMSO-d6, 400.13 MHz): 8.31 (2H, d, J=8.4 Hz, Ho(benzyl)), 7.70 (2H, d, J=8.4 Hz, Hm (benzyl)), 7.64 (d, J=1.6 Hz, 1H, H_{meshyl}), 7.38 (d, J=1.6 Hz, 1H, H_{meshyl}), 7.19 (s, N—CH—CH—, 1H), 7.07 (s, N—CH—CH—, 1H), 6.46 (d, J=16 Hz, 1H, CH₂), 5.81 (d, J=16 Hz, 1H, CH₂), 4.78-4.73 (m, COD, 1H), 4.57-4.51 (m, COD, 1H), 2.95 (m, COD, 2H), 2.12-2.00 (m, COD, 1H), 1.93-1.80 (m, COD, 1H), 1.88 (s, CH₃, 3H), 2.43-2.36 (m, CH₃, 6H), 1.75-1.36 (m, COD, 6H).

¹³C NMR (DMSO-d6, 100.16 MHz): 162.2 (CO), 146.6 (carbene), 138.3, 136.2, 136.1, 134.5, 131.0, 129.2, 128.8, 128.3, 125.1, 124.4, 122.9, 96.3 (COD), 95.1 (COD), 68.0 (d, COD, J_{C-Rh}⁼14 Hz), 67.5 (d, COD, J_{C-Rh}⁼14 Hz), 53.8 (CH₂), 32.7, 32.0, 28.1, 28.0, 27.7, 20.8 (CH₃), 19.4 (CH₃), 17.6 (CH₃).

¹⁹F NMR (DMSO-d6, 376.50 MHz): −154.6-−154.7 (m, 2F, Far), −158.6 (t, J=23.1 Hz, 1F, Far), −163.3-−163.4 (m, 2F, Far).

### Example 9

### Preparation of a compound of formula (I-2) (1-benzyl-3-(4-nitrophenyl)imidazolidene)(cyclooctadiene)rhodium(+1) iodide

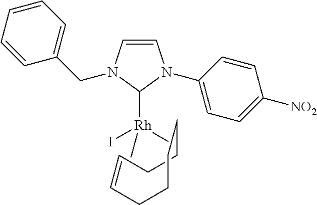

Route A

The compound of Example 1 (44 mg, 0.122 mmol.) and silver oxide (44 mg, 0.061 mmol.) was stirred in 4 ml of degassed methylene chloride at room temperature for 2 h in the dark. The resultant mixture was filtered over a pad of celite. [Rh(COD)Cl]₂ (30 mg, 0.061 mmol.) and KI (50 mg, 0.30 mmol) were then added. The solution was stirred 18 h. at room temperature. Volatiles were removed under reduced pressure and the residue was purified by silica gel chromatography using methylene chloride as elution phase. After concentration, heptane was added until precipitate appeared. The product was recovered by filtration and dried under vacuum. 44 mg (58%) of (1-benzyl-3-(4-nitrophenyl)imidazolidene)(cyclooctadiene)rhodium(+1) iodide was then obtained as an orange powder.

Route B The compound of Example 2 (10 mg, 0.019 mmol.) and KI (10 mg, 0.06 mmol) were stirred 20 h at room temperature in 1 ml of degassed methylene chloride in the dark. The volatile materials were removed under reduced pressure and the residue was purified by silica gel chromatography using methylene chloride as elution phase. After concentration, heptane was added until precipitate appeared. The product was recovered by filtration and dried under vacuum. 9 mg (77%) of (1-benzyl-3-(4-nitrophenyl)imidazolidene)(cyclooctadiene)rhodium(+1) iodide was then obtained as orange powder.

¹H NMR (CDCl₃, 400.13 MHz): δ 8.67-8.65 (2H, m, H(nitrobenzyl)), 8.45-8.41 (2H, m, H(nitrobenzyl)), 7.48-7.38 (5H, m, H(benzyl)), 7.28-7.26 (1H, m, H(imidazolidene backbone)), 6.91 (1H, d, J=2.0 Hz, H(imidazolidene backbone)), 6.00 (1H, m, CH₂), 5.80 (1H, m, CH₂), 5.36-5.28 (2H, m, CH(COD)), 3.38 (1H, br, CH(COD)), 2.74 (1H, br, CH(COD)), 2.33-2.20 (1H, m, CH₂(COD)), 2.19-2.05 (2H, m, CH₂(COD)), 1.93-1.75 (2H, m, CH₂(COD)), 1.71-1.42 (3H, m, CH₂(COD))

¹³C NMR (CDCl₃, 100.16 MHz): δ 185.6 (d, J_{C-Rh}=49.6 Hz, C(Carbene)), 146.7, 145.1, 135.4, 129.1 (CH(benzyl)), 128.7 (CH(benzyl)), 128.5 (CH(benzyl)), 124.6 (CH(nitrophenyl)), 124.6 (CH(nitrophenyl)), 124.2 (CH(nitrophenyl)), 122.5 (CH(imidazolidene)), 121.3 (CH(imidazolidene)), 97.4 (d, J_{C-Rh}=7.0 Hz, CH(COD)), 96.7 (d, J_{C-Rh}=7.0 Hz, CH(COD), 72.1 (m, CH(COD), 56.0 (CH₂), 32.3 (CH₂(COD)), 31.5 (CH₂(COD)), 29.2 (br, m, CH₂(COD)), 28.0 (CH₂(COD)).

MS (ES⁺) calcd: 490.1 (M-I⁻)⁺, 382.0 (M-COD-I)⁺, 640.0 (M+Na)⁺, 1107.1, 1108.1 (2M-I)⁺ Found: 490.2, 382.0, 640.0, 1107.0, 1107.8.

HPLC: t_{R}=8.2 min.

### Example 10

### Radiolabelling Kinetic Studies^{a}

| | Reaction time (min.) | | | | | |
|---|---|---|---|---|---|---|
| | 5 | 10 | 15 | 30 | 45 | 60 |
| Room | — | — | 1.1 ± 0.1 | 12.3 ± 2.2 | 12.1 ± 0.85 | 18.6 ± 3 |
| temperature | | | | | | |
| (% Reaction | | | | | | |
| yield) | | | | | | |
| 37° C. | — | — | 23.4 ± 5.2 | 26.6 ± 4.0 | 74.9 ± 0.3 | 76.1 ± 1.8 |
| (% Reaction | | | | | | |
| yield) | | | | | | |
| 60° C. | 27.2 ± 0.3 | 73.3 ± 0.1 | 88.1 ± 5.4 | 86.8 ± 2.9 | 88.8 ± 0.3 | 83.6 ± 5.0 |
| (% Reaction | | | | | | |
| yield) | | | | | | |
| 100° C. | 95.1 ± 2.1 | 17.9 ± 2.2 | — | — | — | — |
| (% Reaction | | | | | | |
| yield) | | | | | | |

^{a} 1) At-211 (10 μL, 0.7-1.1 MBq), cysteine (8 μl, 7 mg/ml in aqueous solution), vortex 30 s.

2) compound of Example 2 (3 mg/ml in CH₃CN), heating.

The results show that high radiolabelling yields were obtained shortly, over 15 min at 60° C. (88%) and over 5 min at 100° C. (95%). A longer reaction time was needed at 37° C. to obtain acceptable yields (60 min., 76%). Good yields (up to 77%) were obtained for more than 0.5 nmol/μl of astatine solution.

The radiolabelling reaction is significantly shorter than the radionuclide half-life.

### Example 11

### Stability Tests

The compound of Example 4 (7.5-10.5 MBq) was prepared according to the previously described procedure. The resulting organic solution was evaporated until the final volume was about 50 μL. The complex was purified by silica gel chromatography using DCM/MeOH (99/1) as mobile phase. Activity was counted for each 500 μL aliquots and fractions of interest were concentrated under nitrogen flux. One ml of human serum was then added. The solution was gently stirred and divided into two aliquots incubated 15 h. respectively at 4° C. and 37° C. Then, serum proteins were precipitated by adding 750 μl of organic solvents mixture (ACN/DCM 100/1). Supernatant was separated by centrifugation at 4000 rpm and controlled by TLC.

No degradation was observed either at 4° C. than at 30° C., which demonstrates in vivo chemical and enzymatic stability.

1. A compound having formula (I): 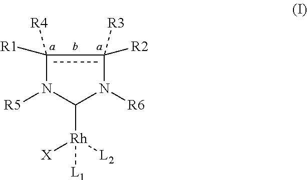
in which:
b is either a single or a double bond;
when b is a double bond, then a is none, R3 and R4 are none and, R1 and R2 are independently chosen from the group consisting of:
H,
ORa,
COORa,
NRaRb,
CONRaRb,
halogen,
NO₂,
CN,
SRa,
COSRa,
PRaRb,
O—P(O)(ORa)₂
P(O)(ORa)₂
(C₁-C₁₀)alkyl, which may be substituted by at least one possibly substituted (C₅-C₁₀)aryl or possibly substituted (C₅-C₁₀)heteroaryl,
(C₅-C₁₀)aryl or (C₅-C₁₀)heteroaryl which may be substituted by at least one possibly substituted (C₁-C₁₀)alkyl, and
functional groups being able to bind a vector, and functional groups having targeting properties,
or, R1 and R2 may form together with the carbon atoms carrying them a (C₅-C₁₀)cycloalkenyl, a (C₅-C₁₀)heterocycloalkenyl which may be substituted by at least one (C₁-C₁₀)alkyl and/or a CO group;
when b is a single bond, then a is a single bond and R1, R2, R3 and R4 are independently chosen from the group consisting of:
H,
ORa,
COORa,
(C₁-C₁₀)alkyl, which may be substituted by at least one possibly substituted (C₅-C₁₀)aryl or possibly substituted (C₅-C₁₀)heteroaryl,
(C₅-C₁₀)aryl or (C₅-C₁₀)heteroaryl which may be substituted by at least one possibly substituted (C₁-C₁₀)alkyl, and
functional groups being able to bind a vector, and functional groups having targeting properties;
R5 and R6 are independently chosen from the group consisting of:
(C₁-C₁₀)alkyl, which may be substituted by at least one possibly substituted (C₅-C₁₀)aryl or possibly substituted (C₅-C₁₀)heteroaryl,
(C₅-C₁₀)aryl or (C₅-C₁₀)heteroaryl, which may be substituted by at least one possibly substituted (C₁-C₁₀)alkyl;
wherein the (C₅-C₁₀)aryl, (C₅-C₁₀)heteroaryl and (C₁-C₁₀)alkyl groups of R5 and/or R6 are possibly substituted by at least one substituent chosen from the group consisting of:
ORa,
COH,
COORa,
NRaRb,
CONRaRb,
halogen,
NO₂,
CN,
SRa,
COSRa,
PRaRb,
O—P(O)(ORa)₂, and
P(O)(ORa)₂
wherein the (C₅-C₁₀)aryl, (C₅-C₁₀)heteroaryl, and (C₁-C₁₀)alkyl of R1, R2, R3, R4, R5 and R6 are possibly substituted by functional groups being able to bind a vector, and functional groups having targeting properties;
and wherein Ra and Rb are independently chosen among H, (C₅-C₁₀)aryl or (C₁-C₁₀)alkyl;
X is a heavy halogen chosen from the group consisting of: ¹²⁵I, ¹²³I, ¹²⁴I, ¹³¹I and ²¹¹At;
L₁ and L₂ are independently chosen from the group consisting of: 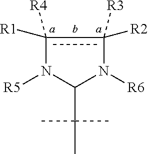
wherein R1, R2, R3, R4, R5, R6, and a, b are defined as above,
possibly substituted (C₅-C₁₀)heteroaryl,
CO group,
PRdReRf, in which Rd, Re and Rf are independently chosen from possibly substituted (C₁-C₁₀)alkyl and possibly substituted (C₅-C₁₀)aryl, and
possibly substituted monocyclic, polycyclic or acyclic (C₂-C₁₀)monoalkene,
or L₁ with L₂ may form together a possibly substituted monocyclic, polycyclic or acyclic (C₆-C₁₀)dialkene,
and the pharmaceutically acceptable salts or bases thereof.

2. The compound according to claim 1 having the formula (I-1): 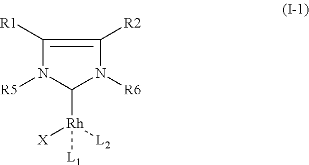
in which R1, R2, R5, R6, L1, L2 and X are defined in claim 1.

3. The compound according to claim 1 wherein R1 and R2 are chosen among:
H,
NO₂,
a substituted (C₁-C₁₀)alkyl,
or, R1 and R2 may form together with the carbon atoms carrying them a (C₅-C₁₀)cycloalkenyl or a (C₅-C₁₀)heterocycloalkenyl possibly substituted by at least one (C₁-C₁₀)alkyl and/or a CO group.

4. (canceled)

5. The compound according to claim 1, wherein R5 is a (C₁-C₁₀)alkyl substituted by a possibly substituted (C₅-C₁₀)aryl.

6. The compound according to claim 5, wherein R5 is a benzyl group.

7. The compound according to claim 1, wherein R6 is chosen among a possibly substituted (C₅-C₁₀)aryl, (C₅-C₁₀)heteroaryl and a (C₁-C₁₀)alkyl.

8. The compound according to claim 7, wherein R6 is a 4-nitrophenyl.

9. The compound according to claim 1, wherein L₁ and L₂ may form together a possibly substituted monocyclic, polycyclic or acyclic (C₆-C₁₀)dialkene.

10. The compound according to claim 9, wherein L₁ together with L₂ form a 1,5-cyclooctadiene.

11. The compound according to claim 1 wherein the functional groups are chosen in the group consisting of:

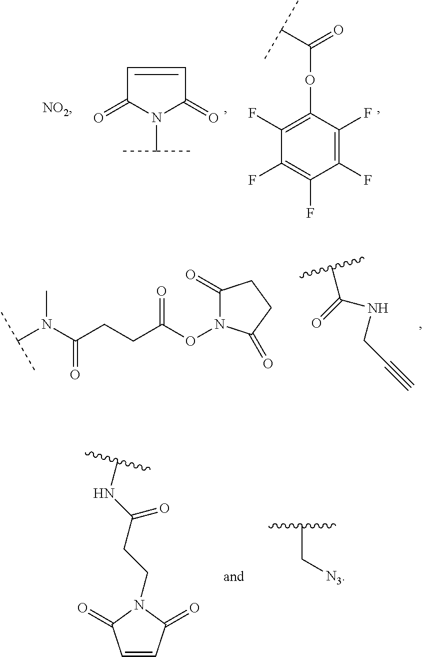

12. The compound according to claim 1, wherein X is ²¹¹At.

13. The compound according to claim 1 wherein at least one of R1, R2, R3, R4, R5 and R6 comprises a functional group being able to bind a vector.

14. The compound according to claim 1, having one the following formulae:

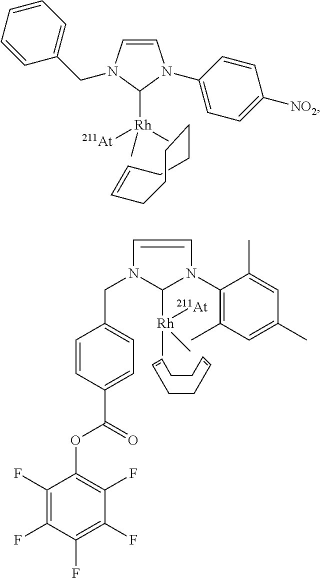

15. A conjugate comprising at least one compound according to claim 13 or 14, covalently linked to a vector through the functional group of said compound.

16. The compound having the formula (II): 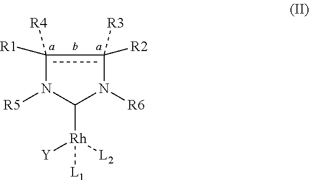
wherein a, b, R1, R2, R3, R4, R5, R6, L₁ and L₂ are as defined in claim 1 and Y is a halogen atom.

17. A method for the preparation of a compound having formula (I) according to claim 1, comprising a step of radiolabelling of a compound of formula (II) according to claim 16.

18. The method according to claim 17, wherein the step of radiolabelling comprises the ligand substitution of Y in a compound of formula (II) according to claim 16 with X at the (—I) oxidation state, X being defined in claim 1.

19. A pharmaceutical composition comprising a compound according to claim 1, in association with at least one pharmaceutically acceptable excipient.

20. A method of localisation of tumors comprising the administration to a patient of a compound having formula (I) according to claim 1 and wherein said compound of formula (I) is administered alone or coupled to a vector.

21. A method of treatment of tumors comprising the administration to a patient of a compound having formula (I) according to claim 1 and wherein said compound of formula (I) is administered alone or coupled to a vector.
